SEQUENCE LISTING (7 sequences)

SEQ ID NO: 1 - protein, 15 aa, Artificial Sequence
  Description of Artificial Sequence Synthetic peptide
CYAAAQWDFGNTMCQ

SEQ ID NO: 2 - protein, 21 aa, Artificial Sequence
  Description of Artificial Sequence Synthetic peptide
CMDYQVSSPIYDINYYTSEPC

SEQ ID NO: 3 - protein, 21 aa, Artificial Sequence
  Description of Artificial Sequence Synthetic peptide
YYTSEPCQKINVKQIAARLLP

SEQ ID NO: 4 - protein, 21 aa, Artificial Sequence
  Description of Artificial Sequence Synthetic peptide
CSSHFPYSQYQFWKNFQTLKC

SEQ ID NO: 5 - protein, 15 aa, Artificial Sequence
  Description of Artificial Sequence Synthetic peptide
FQEFFGLNNCSSSNR

SEQ ID NO: 6 - protein, 9 aa, Artificial Sequence
  Description of Artificial Sequence Control peptide
VQGEESNDK

SEQ ID NO: 7 - protein, 14 aa, Artificial Sequence
  Description of Artificial Sequence Synthetic peptide
CYAAQWDFGNTMCQ

(19) United States
(12) Patent Application Publication
(10) Pub. No.: US 2003/0003440 A1
(43) Pub. Date: Jan. 2, 2003

(54) Novel CCR5 epitope and antibodies against it

(76) Inventor: Lucia Lopalco, Milano (IT)

Correspondence Address: NIXON & VANDERHYE P.C., 8th Floor, 1100 North Glebe Road, Arlington, VA, 22201, US

(21) Appl. No.: 09/805,375

(22) Filed: Mar. 14, 2001

(51) Int. Cl. C12Q 1/70; G01N 33/53; A61K 39/21; C07K 2/00; C07K 4/00; C07K 5/00; C07K 7/00; C07K 14/00; C07K 16/00; C07K 17/00; A61K 38/00; A61K 38/04; C12N 5/06; C12N 5/16

(52) U.S. Cl. 435/5; 424/188.1; 435/345; 435/7.1; 436/548; 530/300; 530/326; 530/389.1

(57) ABSTRACT

The present invention relates to a novel antigenic/immunogenic peptide derived from the CCR5 chemokine receptor, useful in the treatment of HIV infection.

The present invention relates to a novel antigenic/immunogenic peptide derived from the CCR5 chemokine receptor, useful in the treatment of HIV infection.

During the last few years, documentary evidence of the existence of individuals who remain HIV-seronegative and are apparently uninfected despite multiple exposures to HIV-1 (exposed seronegative, ESN) has been produced by a number of groups, suggesting that it is possible to achieve some degree of resistance to the virus. Recently, it was shown that the cells of some ESN couldn't be infected in vitro by M-tropic (R5) strains of HIV because they lack the essential CCR5 coreceptor (Liu, R. et al., 1996, Cell 86:1; Paxton, W. A. et al., 1996, Nature Med. 2:412). In the CCR5 gene, at least two mutations (Quillent, C., 1998, The Lancet 351:1421) have been associated with total or partial resistance to infection by M-tropic R5 strains of HIV, and one mutation was associated with slowing progression of the disease (Kostrikis L., 1998, Nature Med. 4:350). R5 strains (Berger, E. A. et al., Nature 391:240) account for most of the transmission of HIV infections (particularly sexually transmitted infections) and are associated with the earlier phases of the disease (Blaak, H., et al., 1998, J. ViroL. 72:218.).

It was also reported that CCR5 can act as an alloantigen in CCR5-Δ32 homozigous individuals eliciting Abs that compete with RANTES and inhibit infection by R5 primary isolates of HIV-1 (Ditzel, H. J. et al., 1998, Proc. Nat. Acad. Sci. (USA) 95:5241). Furthermore, therapeutic startegies aimed at preventing HIV-1 infection by means of Abs to CCR5 elicited via immunization with a modified CCR5 gene are currently being developed (Zuber, B. et al., Third European Conference on AIDS Research Munich, Germany, February 28 March-3 April 1998 (Abstract 6.S1.)).

A recent report showed that autoantibodies to CCR5 could be induced in C57BL/6 mice by inoculation with a papilloma virus modified to express CCR5 peptides (Chackerian, B. et al., Proc. Nat. Acad. Sci. (USA) 96:2373). Such Abs could inhibit binding of b-chemokines to CCR5, as well as block infection with HIV-1.

The present inventors investigated the possibility that sera from ESN, their HIV-infected sexual partners (HIV+), and healthy controls (USN), contained CCR-5 specific Abs, studying whether incubation of PBMC with sera could prevent macrophage inflammatory protein 1β (Mip 1β) (natural ligand of CCR5) binding to CCR5. Whereas sera from either controls or HIV-infected individuals could not interfere with the binding of Mip 1β to CCR5, inhibitory activity was surprisingly found in sera of a number of ESN. The results of these studies are thoroughly illustrated in Journal of Immunology, 2000, vol. 164, 3426-3433, which is herein incorporated by reference in its entirety.

Characterisation of this inhibitory activity indicated that the anti-CCR5 Abs present in the sera of some ESN, down modulate CCR5 expression on PBMC in vivo, inhibit Mip1b-induced chemotaxis of control PBMC and block the HIV coreceptor function of CCR5, neutralizing the infectivity of R5 strains of HIV-1. Further investigation was carried out to determine the epitope of CCR5 recognised by the specific antibodies present in ESN sera. Accordingly, the latter were tested on a panel of synthetic peptides (see below) covering the complete sequence of the extra membrane region of CCR5.

Specific Ab binding to the peptide CYAAAQWDFGNTMCQ, corresponding to the second external domain of CCR5 (first cysteine loop—aa 89-102) modified by addition of a Cys residue at the N-terminus, was observed. Anti-CCR5 Abs revealed highly selective for this epitope, as no binding was observed on a panel of other peptides (peptides 1,2,4 and 5, see Table).

Importantly, the Cys residue introduced at the N-terminus resulted critical for anti-CCR5 antibodies recognition, as the peptide binding of such antibodies was abolished by addition of 2-mercaptoethanol and N-ethyl maleimide, which cause reduction and alkylation of the cysteine loop. This suggests that the peptide has to satisfy specific conformational requirements in order to provide an effective binding to anti-CCR5 antibodies from ESN sera. Although the CCR5 epitope used by Chackerian et al. (supra) to immunize mice and the epitope herein disclosed show more than 90% homology, it could not be predicted that the addition of a cysteine residue and the consequent internal cyclization of the peptide would increase its binding to anti-CCR5 antibodies naturally occurring in the sera of subjects resistant to HIV infection. Furthermore, the same peptide in the correct conformation was shown to possess a marked immunogenic activity in mice.

The present invention is therefore directed to a peptide of sequence CYAAQWDFGNTMCQ (SEQ ID N. 1) and to a method for the treatment of patients infected by HIV or of subjects exposed, or at risk of exposure, to HIV, which comprises administering to said patients or subjects an effective amount of the same peptide in order to elicit an immune response against it, or alternatively administering antibodies raised to that peptide to prevent or inhibit HIV infection/progression.

The peptide is preferably prepared synthetically, for example according to the procedures described in Merrifield, (1986) Science 232:341-347, and Barany and Merrifield, The Peptides, Gross and Meienhofer, eds (N.Y., Academic Press), pp. 1-284 (1979). The synthesis can be carried out in solution or in solid phase or with an automatized synthesiaer (Stewart and Young, Solid Phase Peptide Synthesis, 2^{nd} ed., Rockford Ill., Pierce Chemical Co., 1984). Alternatively, the recombinant DNA technology can be used.

One or more amino acids, corresponding or not to the original protein sequence of h-CCR5, can be added to the amino or carboxy terminus of the 14-meric peptide of the invention, provided that such a modification does not impair the formation of a disulphide bond between the Cys residues in position 1 and 13 of SEQ ID N. 1, which was proved by the inventors to confer the correct immunogenic conformation to the epitope. Further modifications include amidation or esterification of the terminal carboxyl, addition of lipophilic groups (e.g. myristyl), glycosylation or conjugation with other peptides, or other means for coupling the peptide to another protein or peptide molecule or to a support, e.g. to increase immunogenicity or higher bioavailability after administration.

The peptide or its derivatives will preferably be administered in form of vaccine. A vaccination protocol can comprise active or passive immunization, whereby active immunization entails the administration of the peptide or its derivatives to the host/patient to elicit a protective immune response, whereas passive immunization entails the transfer of preformed immunoglobulins or fragments thereof to a host/patient. Theory and practice of vaccination and vaccines are known to anyone skilled in the art, see, for example, Paul, “Fundamental Immunology” Raven Press, New York (1989) or Cryz, S. J., “Immunotherapy and vaccines”, VCH Verlagsgesselshaft (1991). Vaccines are conventionally prepared in the form of injectables, suspensions or solutions, but they can also be used in the form of solid preparations or liposomes. The immunogenic ingredients can be mixed with pharmacologically acceptable excipients, such as emulsifiers, buffering agents and adjuvants which increase the efficacy of the vaccine. The latter can be administered according to single or multiple dosage schedule. Multiple dose provides 1 to 10 separate doses, each containing a quantity of antigen varying from 1 μg to 1000 μg, preferably from 5 to about 250 μg, followed by further doses at subsequent time intervals, necessary to maintain or to reinforce the immune response and, if required by the subject, a further dose after several months. In any case the treatment regimen will depend on the response elicited in the treated patient and non his general health conditions. It is however noted that vaccine strategies aimed at elicitation of anti-CCR5 Abs could be less dependent on the need for repeated vaccine boost, compared with vaccine strategies based on the induction of HIV-specific immune responses.

In a further aspect, the invention relates to a preparation of substantially isolated monoclonal or polyclonal antibodies specifically immunoreactive with the peptide of the invention. Such antibodies may be used as protective agents against HIV infections, for example in passive immunization as above described. They can be produced by immunizing an experimental animal with the peptide and then isolating the specific antibodies. Techniques for producing and processing polyclonal antibodies are known in the art and are described for example in Mayer and Walker eds., “Immunochemical Methods in Cell and Molecular Biology”, Academic press, London (1987). Methods for purifying antibodies are known in the art and comprise, for example, immunoaffinity chromatography. The antibodies obtained from the animal may be further manipulated, e.g. cleaved to obtain suitable fragments or may be the starting compounds for genetically engineered antibodies or derivatives thereof, such as scFv or Fab fragments having the same binding specificity. Furthermore, the antibodies or their derivatives may be used to produce anti-idiotypic antibodies that mimic the CCR5 epitope. The demonstrated ability of anti-CCR5 antibodies according to the invention to prevent chemokine (Mip1β)/receptor binding, suggests their use as anti-inflammatory agents or more generally as chemokine competitors in all those pathologies where a reduction of such receptor/chemokine binding is desired, for example in the graft versus host disease where an increased production of chemokines is observed.

According to a different aspect, the invention relates to a method for detecting an antibody to CCR5, fragments or derivatives thereof, in a sample, which comprises (a) incubating said sample with the peptide of the invention or derivative thereof and (b) detecting the formation of a complex between said antibody and peptide. The peptide(s), antibodies, fragments or derivatives thereof described above are suitable for use in immunoassays, either radioisotopic or non-radioisotopic, comprising, for example, RIA (Radioisotopic Assay) and IRMA (Immune Radioimmunometric Assay), EIA (Enzyme Immuno Assay), ELISA (Enzyme Linked Immuno Assay), FIA (Fluorescent Immuno Assay), and CLIA (Chemioluminescent Immune Assay). For those applications, the peptide, antibodies or derivatives thereof are preferably attached to a solid phase carrier whereon the reaction with the test sample is carried out.

The invention will be further illustrated by the following examples.

### EXAMPLE 1

Synthesis of Peptides and Preparation of Peptide/Beads

Peptides (Table) were synthesized by the solid phase F-moc method (Fields, G. B. et al., 1990, Int. J. Pept. Prot. Res. 35:161) using an Applied Biosystem model 433 A peptide synthetizer. After the peptide assembly, the side chain protected peptidyl resin was de-blocked as previously described (Kings, D. S. et al., 1990, Int. J. Pept. Prot. Res. 36:255) and purified to apparent homogeneity by reverse-phase chromatography. An extra-sequence cysteine was added to peptides 1, 3, and 4, to obtain conformationally cyclic peptides. These peptides were treated overnight with a 5-fold excess of oxidized glutathione and purified by reverse phase chromatography.

Coupling of CCR5 peptides to tosyl-activated Dynabeads M280 (Dynal, Oslo, Norway) was obtained following standard procedures. Briefly, 3×10⁷ beads were incubated with 9 μg of CCR5 peptides in 50 mM borate buffer pH 9,5 (16 h at 37° C.). After 4 washes in PBS, peptide/beads were ready for use.

TABLE
Peptides covering the sequence of the extramembrane region of CCR5

| Peptides | Sequence^{a} |
|---|---|
| 1 | cMDYQVSSPIYDINYYTSEPC |
| 2 | YYTSEPCQKINVKQIAARLLP |
| 3 | cYAAAQWDFGNTMCQ |
| 4 | CSSHFPYSQYQFWKNFQTLKc |
| 5 | FQEFFGLNNCSSSNR |

^{a}Amino acids in capital letters were introduced to obtain conformationally peptides.

### EXAMPLE 2

Immunoglobulin Purification

Anti-Human polyvalent immunoglobulin -coupled Agarose (Sigma-Aldrich) was utilized to purify total Ig from the sera of ESN (HIV-exposed individuals) and USN (healthy controls). Briefly, 100 μl of serum were incubated overnight at 4° C. in columns containing 5 ml of anti-human IgG-agarose. After recovering the column washout (Ig-depleted fraction), the columns were washed six times in phosphate buffer (0.01M with 0.5M NaCl). Bound Ig's were eluted with glycine/NaCl 0.2M, and the eluted fractions neutralized with 1M Tris (Ig-enriched fraction). Ig-enriched and Ig-depleted fractions were concentrated on Ultrafree-15 Biomax 30 membranes (Millipore, Bedford, USA) with a cut off of 30 kDa and dialyzed against RPMI. Ig concentration was determined by ELISA using commercial Ig's as standard, and adjusted to 2.5 mg/ml (corresponding to a serum dilution of 1:10). The Ig-depleted fractions were diluted by the same factor.

Affinity-purification of Antibodies on Peptide/Beads

Binding of anti-CCR5 specific Igs to peptide/beads was obtained by incubating 9 μg Ig's to 9 μg peptide/beads for 1 h at 4° C. Ig's were eluted in 0.5 M acetic acid, dialyzed against RPMI medium, then tested in Mip1β binding assays and/or in HIV neutralization assays. To establish if the region recognized by anti-CCR5 antibodies corresponds to a conformational epitope, the specific peptide/beads were incubated with 10 mM of βME, and subsequently with 300 mM of N-Ethyl Maleimide (final concentration: 30 mM) for 60 min prior to antibody binding.

### EXAMPLE 3

Mip1β Binding Assay

The assay was performed as described (Trkola, A. et al., 1996, Nature 384:184). Briefly, 10⁶ purified CD4⁺ cells in 200 μl of RPMI (Gibco-Life Technologies, Milan, Italy)(containing 0.05 M NaN₃, 1% BSA and 25 mM HEPES) were incubated with appropriate dilutions of affinity-purified anti-CCR5 Abs (Example 1); after 45 min of incubation ¹²⁵I-Mip1β (Dupont-NEN, Mechelem, Belgium) was added (final concentration 0.1 nM, 0.2 μCi), and the cells were further incubated for 2 h on ice. Unbound radioactivity was separated by centrifugation on a two-step gradient (Grassi, F. et al., 1991, J. Exp. Med. 174:53) in 0.3 ml tubes (Nunc, Roskilde, Denmark) as follows: the lower layer consisted of fetal calf serum (FCS) containing 10% sucrose; the upper layer consisted of 80% silicone (Sigma-Aldrich) and 20% mineral oil (Sigma-Aldrich). The bound radioactivity in the cell pellets was measured in a gamma counter. Serum samples were diluted 1:10 (5 replicas for each sample). A specificity control consisting of a 100-fold excess of unlabeled Mip1β was included in all experiments. The binding of the ¹²⁵I-Mip1β to activated CD4⁺ cells ranged between 1000 and 6000 CPM. The cut-off value was set at 12% (3 SD above the mean value of the 45 USN serum samples).

Results

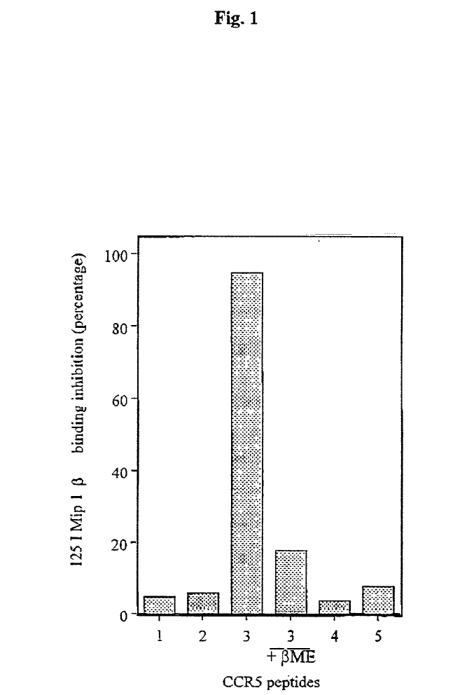

FIG. 1 reports the epitope mapping of anti-CCR5 Abs assayed in the Mip1β binding inhibition assay. Specific Ab binding to peptide 3 (aa 89-102), corresponding to the second external domain (first cysteine loop) of CCR5, was observed. As shown in the Figure, the Abs are highly selective for this epitope as no binding was observed on a panel of other peptides (peptides 1, 2, 4 and 5). Binding of the anti-CCR5 Abs to peptide 3 was abolished by addition of 2-ME and N-ethyl maleimide.

### EXAMPLE 4

Virus Neutralization Assays

The “resting cell assay” was performed according to Zolla-Pazner (Zolla Pazner, S. et al., 1995, AIDS Res. Hum. Retovir. 11:1449). Briefly, 2×10⁵ resting PBMC were added to 75 μl of serial dilutions of Ig-enriched fractions from ESN or USN; after 1 h incubation, 75 μl of a virus dilution (ID₅₀ adjusted to 20) was added. The cultures were incubated for two more hours, washed and resuspended in PHA and IL-2-containing medium. HIV-1 p24 antigen in the supernatants was determined on days 7 and 9. Percent neutralization was calculated relatively to a non-treated control.

For the “activated PBMC assay”, the cells were cultured in medium containing PHA and IL2 for 48 h prior to the neutralization assay.

Affinity-purified Anti-CCR5 Antibodies Inhibit HIV-1 Replication

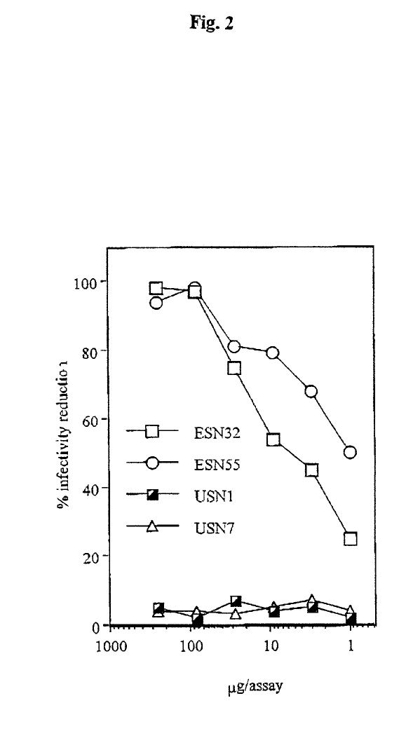

Antibodies to Pep 3 were affinity-purified from two ESN sera and tested in HIV neutralization assays. Neutralization of HIV-1 primary isolates (HIV36) is shown in FIG. 2. The neutralizing titers obtained with Ig's eluted from Pep 3 were higher (IC₅₀ of 1 μg/ml for ESN 55 and 5 μg/ml for ESN 32) that those of total serum immunoglobulins (IC₅₀ of 13 μg/ml for ESN 55 and 82.7 μg/ml for ESN 32).

### EXAMPLE 5

Generation of Mouse Serum Against Peptide 3from CCR5

BALB/c mice were immunized i.p. every 15 days with peptide 3 or with an unrelated control peptide (VQGEESNDK); both peptides had previously been conjugated to KLH (50 μg/dose). Serum was monitored for the presence of antibodies against the immunogens. After three immunizations the mice were sacrificed and sera were tested in direct binding on pep 3.

Binding of Mice Sera to Peptide 3 Conjugated to Dynabeads

Binding of mice sera to peptide 3 was obtained by ELISA. Microwells plates were coated with Beads/pep 3 (10⁷beads). The plates were satured for 1 h with PBS and 3% BSA. Different dilutions of mice antisera (1/10, 1/50, 1/250 and 1/1000) were added and incubated for 2 h at r.t. Mouse Ig binding was revealed with HRP conjugated rabbit anti mouse Ig (Dako, Santa Barbara, Calif.). The enzimatic reaction was developed and read at 492 nm.

Peptide 3 Mice Serum Specifically Recognise the CCR5 Peptide

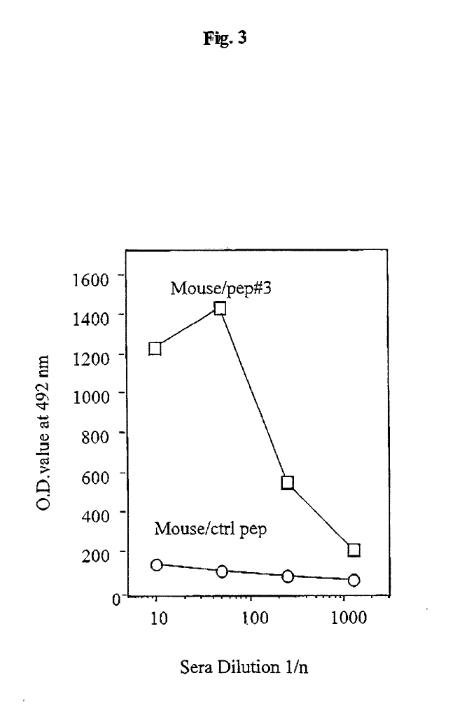

BALB/c mice were immunized with either peptide 3 or a control peptide. Peptide 3 specific sera were then tested in direct binding on pep 3 by ELISA. As shown in FIG. 3, a specific binding on pep 3 was observed when sera of mice immunized with peptide 3 was used. In contrast no binding was detected when sera of mice immunized with the control peptide was tested in the assay.

### EXAMPLE 6

Anti CCR5 Antibodies Inhibit Biological Function of CCR5

PBMC from one healthy donor (selected for high expression of CCR5) were activated with PHA and IL2 for 3 days (see above) in the presence of two concentrations of purified Ig's (250 and 62 μg/ml) from three ESN (No. 32,34 and 55) and one USN (No. 5, control). 3×10⁵ activated PBMC in 50 μl of RPMI medium containing 0.3% human serum albumin were placed in the upper chamber of 5-μm pore size bare filter Transwell (Costar, Europe, Amsterdam, Netherlands). Chemotaxis was carried out in the presence of 1.5 μg/ml of Mip1β (placed in the lower chamber). The transwells were incubated for 2 h at 37° C.; cells that migrated from the upper to the lower chamber were then quantified by FACS analysis. PBMC from ESN34 and ESN55 were also used in chemotaxis assays to evaluate the capacity of ESN PBMC to migrate in the presence of Mip1β. The results were expressed as chemotaxis index (CI), which represents the fold increase in the number of migrated cells in response to Mip1β over the spontaneous cell migration in control medium.

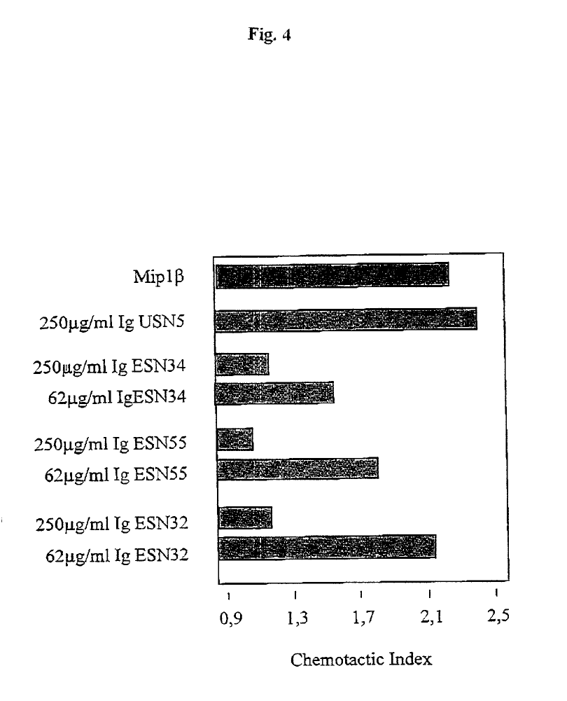

When 250 and 62 μg/ml of anti CCR5 antibodies were incubated with CD4 cells, a strong reduction of Chemotaxis index was observed in a dose dependent manner (shown in FIG. 4). These results demonstrate that anti CCR5 antibodies, present in ESN, interfere with the biological function of CCR5 on lymphocytes from healthy controls.

1. Immunogenic peptide derived from the CCR5 chemokine receptor, having the following sequence: CYAAAQWDFGNTMCQ.

2. Monoclonal or polyclonal antibodies against the peptide of claim 1.

3. A pharmaceutical composition comprising the peptide of claim 1 or the antibodies of claim 2.

4. A pharmaceutical composition containing the peptide according to claim 3, which is in form of vaccine.

5. A method for inducing immunity against the CCR5 protein which comprises administering to a human subject an effective amount of the immunogenic peptide of claim 1 or of the antibodies of claim 2.

6. A method according to claim 3, wherein the subject is a patient infected by HIV or he has been exposed, or is at risk of exposure, to HIV.

7. A method to inhibit or prevent HIV infections which comprises inducing immunity against CCR5 receptor by administering to a patient infected by HIV or to a subject exposed, or at risk of exposure, to HIV, an effective amount of the immunogenic peptide of claim 1 or of the antibodies of claim 2.

8. A method of treating diseases in the etiopathogenesis of which Mip1ξ/CCR5 binding is involved, which comprises administering to a subject in need of such a treatment an antibody according to claim 2.

9. A method according to claim 8, wherein said diseases are selected from inflammation and graft versus host diseases.

10. A method for detecting an antibody to CCR5 in a sample, which comprises (a) incubating said sample with the peptide of claim 1, or a derivative thereof, and (b) detecting the formation of a complex between said antibody and peptide.

11. The use of the antibodies of claim 2 to prevent chemokine Mip1β/CCR5 binding.
